(19) United States
(12) Patent Application Publication
(10) Pub. No.: US 2009/0312700 A1
(43) Pub. Date: Dec. 17, 2009

(54) Device for Minimally Invasive Insertion into a Physiological Lumen

(75) Inventors: Alexander Winter, Rostock (DE); Frank Bakczewitz, Rostock (DE)

Correspondence Address: BIOTECH BEACH LAW GROUP , PC, 625 BROADWAY, Suite 1210, SAN DIEGO, CA, 92101, US

(73) Assignee: BIOTRONIK VI PATENT AG, Baar (CH)

(21) Appl. No.: 12/480,162

(22) Filed: Jun. 8, 2009

(30) Foreign Application Priority Data
Jun. 17, 2008 (DE) 10 2008 002 479.1

(51) Int. Cl. A61M 25/092; H01L 41/22

(52) U.S. Cl. 604/95.05; 29/25.35

(57) ABSTRACT

A device for the minimally invasive insertion into a lumen of a hollow organ comprises a hose-shaped wall (3) with a multiplicity of piezoelectric fibers (4) embedded therein.

### CROSS REFERENCE TO RELATED APPLICATIONS

The present application claims benefit of priority to German patent application no. DE 10 2008 002 479.1, filed on Jun. 17, 2008; the contents of which are herein incorporated by reference in their entirety.

### TECHNICAL FIELD

The invention relates to a device for minimally invasive insertion into a physiological lumen and methods of its production.

### BACKGROUND OF THE INVENTION

Fundamentally, devices for insertion into a physiological lumen are realized as catheters as a rule. Conventional catheter shafts usually comprise a multilayer compound of polymer tubes and/or metal tubes that lie coaxially inside one another. A material compound that appears to be optimal is selected depending on the use. Subsequently, there is no longer any possibility of influencing the mechanics or the geometry of the catheter. Catheters are known from U.S. Pat. No. 5,415,633 and U.S. Pat. No. 6,083,170, which have a control element on their distal end for deforming the same.

### SUMMARY OF THE INVENTION

The object of the present invention is therefore to create a catheter with improved mechanical properties. The core of the invention lies in arranging at least one fiber in the wall of a catheter, the length of which fiber can be regulated in a targeted manner by means of a control device. The mechanical as well as the geometric properties of the catheter can be adjusted to the respective requirements hereby in a targeted and controlled manner.

In one aspect of the present invention, a device for minimally invasive insertion into a physiological lumen is provided. An exemplary device includes a hollow body, which extends at least in some sections along a longitudinal direction, with a wall, and a control device. In various embodiments, the wall has at least one fibrous element, the length of which can be changed in a targeted manner by means of the control device. In some embodiments a multiplicity of fibrous elements are provided.

The at least one fibrous element may be embedded in a polymer matrix and may extend over the entire length of the wall. The at least one fibrous element may be aligned in parallel along the longitudinal direction of the hollow body and/or concentrically to the circumference of the wall of the hollow body. The at least one fibrous element may be embedded completely in the material of the wall and/or may be embedded in the material of the wall in a spiral-shaped manner around a central axis. In some embodiments the fibrous elements are piezoelectric actuators.

The control device may change geometric and/or mechanical properties of the wall.

In related embodiments, a method of producing the device for minimally invasive insertion into a physiological lumen is provided, the method including extruding a wall of a hollow body and simultaneously embedding at least one fibrous piezoelectric element in the wall by means of a coextrusion method.

Features and details of the invention are shown by the following description of an exemplary embodiment based on the drawings.

### BRIEF DESCRIPTION OF THE DRAWINGS

FIG. 1 is a perspective sectional representation through an exemplary embodiment of the invention.

FIG. 2 is a further diagrammatic representation of the exemplary embodiment according to FIG. 1.

FIG. 3 is a diagrammatic sectional representation through the wall of a catheter according to the invention in the area of the fibers adjustable in length.

### DETAILED DESCRIPTION OF PREFERRED EMBODIMENTS

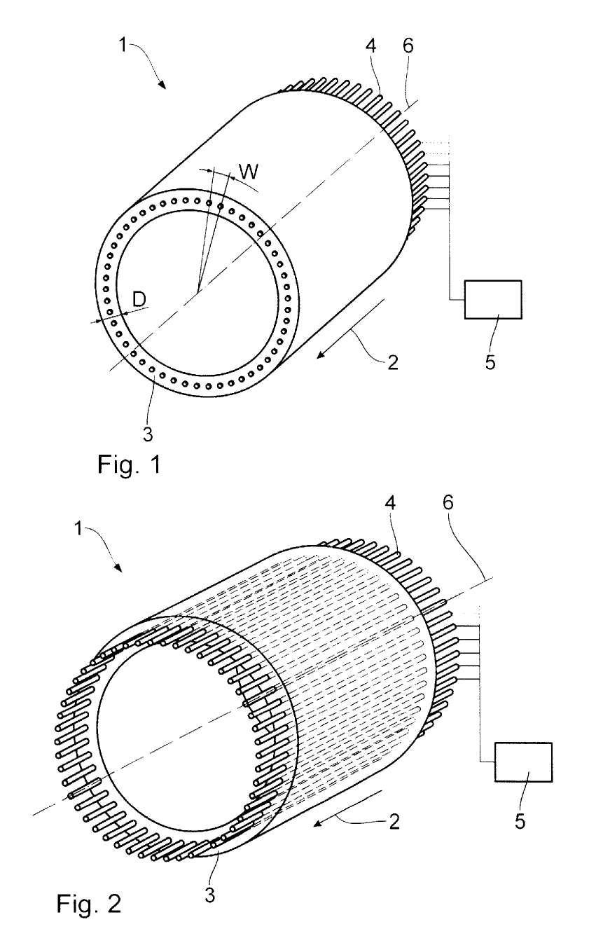

An exemplary embodiment of the invention is described below with reference to FIGS. 1 through 3. The device comprises a hose-shaped hollow body 1, which extends at least in some sections along a longitudinal direction 2. The hollow body 1 is embodied in particular in a hollow cylindrical manner and comprises a wall 3 with a thickness D. The thickness D lies in the range of 100 μm to 1 mm. The wall 3 comprises a composite material, in particular a fibrous composite material. It comprises at least in part a polymer plastic, which forms a bedding matrix. The wall 3 can be embodied in particular in a multilayer manner.

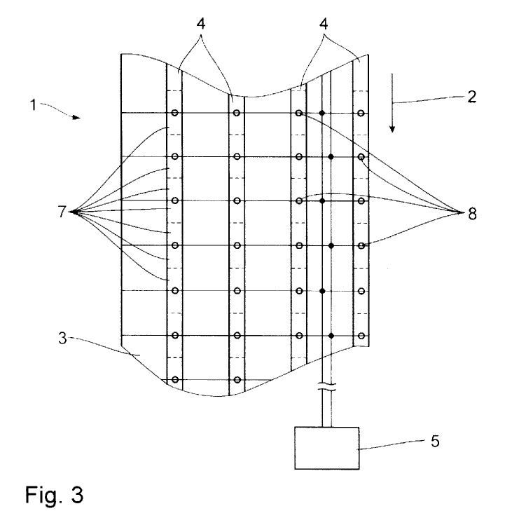

The wall 3 is flexible, preferably elastically deformable and has an annular cross section in the initial state. However, other cross sections are likewise conceivable. A multiplicity of piezoelectric fibers 4 are embedded in the polymer matrix of the wall 3. The piezoelectric fibers 4 serve as an example of fibrous elements, the length of which can be changed in a controlled manner by means of a control device 5 shown in FIGS. 1 and 2 only in a diagrammatic manner. The control device 5 is part of the device according to the invention. The geometric and/or mechanical properties of the wall 3 can hereby be changed in a targeted manner by means of the control device 5. The piezoelectric fibers 4 comprise, for example, lead zirconate titanate.

According to the embodiment shown in the figures, a multiplicity of piezoelectric fibers 4 aligned parallel to one another are provided in the wall 3. The fibers 4 are aligned parallel to the longitudinal direction 2 and extend in the longitudinal direction 2 over the entire length of the wall 3. The piezoelectric fibers 4 are completely embedded in the polymer matrix of the wall 3. Preferably the piezoelectric fibers 4 are distributed uniformly over the circumference of the wall 3. A non-uniform distribution. e.g., in four packets over the circumference, could also be advantageous. The piezoelectric fibers 4 are arranged at an angular distance W regarding a central axis 6 of the hollow body 1 with respect to one another. The angular distance W hereby lies in the range of 0.1° to 10°.

An arrangement of the piezoelectric fibers 4 concentrically to the central axis 6 alternatively and/or additionally to the piezoelectric fibers 4 in the longitudinal direction 2 is, of course, likewise possible and can be particularly advantageous depending on the application. Accordingly, in an alternative embodiment not shown in the figures it is provided to arrange the piezoelectric fibers 4 in a spiral-shaped manner around the central axis 6.

The number of the piezoelectric fibers 4 lies in the range of 1 to 1000, in particular in the range of 10 to 100, in particular in the range of 20 to 50.

The piezoelectric fibers 4 have a diameter in the range of 5 μm to 500 μm, in particular in the range of 100 μm to 300 μm.

The piezoelectric fibers 4 serve as piezoelectric actuators, which are addressable individually and/or in groups by the control device 5. To this end the control device 5 is connected in an electrically conducting manner to each of the piezoelectric fibers 4. The length of the piezoelectric fibers 4 can be changed in a controlled manner through the application of an electric voltage to the piezoelectric fibers 4 by means of the control device 5. The geometric and/or mechanical properties of the wall 3 can be changed in a targeted manner hereby by means of the control device 5.

The piezoelectric fibers 4 can be divided into a plurality of sections 7 in the longitudinal direction 2, wherein a voltage with adjustable polarity and amplitude can be applied to each individual section 7 independent of the others by means of the control device 5. To this end each piezoelectric fiber 4 has a plurality of electrical connection points 8 with the control device 5.

The mode of operation of the device is described below. The device, which is used in particular as a catheter for the minimally invasive insertion into a lumen of a hollow organ, in the initial state, in which no electric voltage is applied to the piezoelectric fibers 4, is flexible, elastic and at least in some sections aligned along the longitudinal direction 2. The catheter is straight over its entire length, i.e., aligned along the longitudinal direction 2. However, if an electric voltage of a certain polarity is applied to at least one of the piezoelectric fibers 4, this fiber 4 changes its length, for example, becomes longer. This can be used to change the geometry of the catheter. In particular through the change of the length of the piezoelectric fibers 4 in a certain angular range with respect to the central axis 6, which is in particular less than 180°, a curvature of the catheter can be produced in a targeted manner. A lengthening of the fibers 4 hereby leads to a convex surface of the wall 3 in the said angular range with respect to the central axis 6, while a shortening of the fibers 4 leads to a concave or saddle-shaped surface in this angular range. This effect can be intensified through the application of a voltage with reverse polarity to the piezoelectric fibers 4, which are arranged in a second angular section lying opposite the first angular section with respect to the central axis 6.

Accordingly, the flexibility and/or the elasticity or in general the mechanical properties of the catheter can be changed by the application of a voltage of the same polarity and amplitude to piezoelectric fibers 4 respectively lying opposite one another with respect to the central axis 6. In particular it is possible to strengthen the catheter through the application of the same voltage to all of the piezoelectric fibers in a specific section with respect to the longitudinal direction 2.

With the arrangement of at least some fibers 4 concentrically to the central axis 6, furthermore the diameter or circumference of the catheter can also be preferably dynamically changed by the application of a voltage to these fibers 4.

The production of the device according to the invention is described below. First the wall 3 of the hose-shaped hollow body 1 is produced by an extrusion method. The piezoelectric fibers 4 are coextruded hereby, i.e., simultaneously with the extrusion of the wall 3 embedded therein. This leads to a particularly reliable connection between the piezoelectric fibers 4 and the wall 3 of the catheter. Subsequently the control device 5 is connected to the piezoelectric fibers 4. To this end in particular a laser soldering, laser microwelding or a so-called laser droplet welding method is provided.

What is claimed is:

1. A device for minimally invasive insertion into a physiological lumen, comprising:
a) a hollow body (1), which extends at least in some sections along a longitudinal direction (2), with a wall (3); and
b) a control device (5);
wherein the wall (3) has at least one fibrous element (4), the length of which can be changed in a targeted manner by means of the control device (5).

2. The device according to claim 1, characterized in that at least one fibrous element (4) is embedded in a polymer matrix.

3. The device according to claim 1, characterized in that the at least one fibrous element (4) extends over the entire length of the wall (3).

4. The device according to claim 1, characterized in that a multiplicity of fibrous elements (4) aligned parallel to one another is provided.

5. The device according to 1, characterized in that geometric, mechanical or both geometric and mechanical properties of the wall (3) can be changed by means of the control device (5).

6. The device according to claim 1, characterized in that at least one fibrous element (4) is aligned parallel to the longitudinal direction (2).

7. The device according to claim 1, characterized in that at least one fibrous element (4) is aligned concentrically to the circumference of the wall (3) of the hollow body (1).

8. The device according to claim 1, characterized in that at least one fibrous element (4) is embedded in the material of the wall (3) in a spiral-shaped manner around a central axis (6).

9. The device according to claim 1, characterized in that at least one fibrous element (4) is embedded completely in the material of the wall (3).

10. The device according to claim 1, characterized in that the fibrous elements (4) are piezoelectric actuators.

11. The device according to claim 4, characterized in that the length of the fibrous elements (4) can be controlled individually and/or in groups, in particular can be changed by section, by means of the control device (5).

12. A method for producing a device according to claim 1, comprising the following steps:
a) extruding a wall (3) of a hollow body (1); and
b) simultaneous embedding at least one fibrous, piezoelectric element (4) in the wall (3) by means of a coextrusion method.
